(19) United States
(12) Patent Application Publication
(10) Pub. No.: US 2024/0050312 A1
(43) Pub. Date: Feb. 15, 2024

(54) OPHTHALMIC BLISTER PACK

(71) Applicant: TWENTY TWENTY THERAPEUTICS LLC, South San Francisco, CA (US)

(72) Inventors: Timothy Stowe, Alameda, CA (US); Alexandre R. Tumlinson, South San Francisco, CA (US)

(21) Appl. No.: 18/232,307

(22) Filed: Aug. 9, 2023

Related U.S. Application Data
(60) Provisional application No. 63/396,357, filed on Aug. 9, 2022.

(51) Int. Cl. A61J 1/06; A61F 9/00; B65D 75/32

(52) U.S. Cl. CPC A61J 1/067; A61F 9/0008; B65D 75/327; B65D 2575/3245; B65D 2575/3254

(57) ABSTRACT

A blister package for ophthalmic drugs that includes a base layer having a first side and a second side. At least one dome extends from the first side with each dome defining a cavity that receives a liquid drug dose. An intermediate layer extends over the second side of the base layer and covers each cavity. The intermediate layer includes an integrally formed nozzle aligned with each cavity. A removable layer extends over the intermediate layer and is releasable from the intermediate layer for uncovering each nozzle opening.

### RELATED APPLICATIONS

This application claims the benefit of U.S. Provisional Application Ser. No. 63/396,357, filed Aug. 9, 2022, the entirety of which is incorporated herein by reference.

### TECHNICAL FIELD

The present invention relates generally to drug packaging and, in particular, relates to unidose ophthalmic blister packs.

### BACKGROUND

Blister packaging for both solid and gel drugs have been used to help store and dispense said drugs. In some instances, an aluminum layer of the packaging is punctured or otherwise removed to allow access to the drug. These traditional puncture-based packages, however, are not ideal for liquid drug delivery in that portions of the aluminum layer can obstruct or impede the flow of the drug out of the packaging, thereby making drug delivery unreliable and/or difficult.

### SUMMARY

In one example, a blister package for ophthalmic drugs includes a base layer having a first side and a second side. At least one dome extends from the first side with each dome defining a cavity that receives a liquid drug dose. An intermediate layer extends over the second side of the base layer and covers each cavity in the base layer. The intermediate layer includes an integrally formed precision nozzle opening or pattern of nozzle openings aligned with each cavity that is sufficiently small for liquid not to escape unless internal pressure on the dome is exercised. A top peelable layer extends over the nozzle openings for covering each nozzle. The removable layer prevents moisture vapor transmission through the nozzle openings for long term drug storage in the blister pack.

In another example, a blister package for ophthalmic drugs includes a polymer base layer having a first side and a second side. Hemispherical domes extend from the first side with each dome defining a cavity that receives a liquid drug dose. An intermediate layer extends over the second side of the base layer and covers each cavity. The intermediate layer includes an integrally formed nozzle aligned with each cavity and having slits extending entirely through the intermediate layer. A peelable top composite layer can be formed from a “blister foil” layer laminate that incorporates a release lining for easy peel back to expose nozzle openings in the intermediate layer. The blister foil layer can be an aluminum composite layer with aluminum at its core for low water vapor transmission and a thin top printable protective film such as polypropylene (PP) and a bottom protective layer such as polyvinyl chloride (PVC). It can also have a release layer such as PVC or a polyester (PE) film that allows the easy peel functionality.

In another example, a blister package for ophthalmic drugs includes a base layer having a first side and a second side. A nozzle opening extends from the first side towards the second side and a cavity extends from the second side towards the first side and into fluid communication with the nozzle. The cavity is configured to receive a drug dose deposited before layers are sealed together by localized heat welding or adhesive. A top removable seal layer extends over the first side of the base layer and covers the nozzle openings. A second seal layer extends over the second side of the base layer and forms the cavity for liquid drug deposition. The top outside seal layer is removable from the base layer to uncover the nozzle. Applying force to the dome feature towards the nozzle openings urges the drug dose through the nozzle openings and out of the package when the top outside layer has been peeled off.

Other objects and advantages and a fuller understanding of the invention will be had from the following detailed description and the accompanying drawings.

### BRIEF DESCRIPTION OF THE DRAWINGS

FIG. 1 is a schematic illustration of an example blister package in accordance with an aspect of the present invention.

FIG. 2 is a section view of the package of FIG. 1 taken along line 2-2.

FIG. 3 is a schematic illustration of a method of forming the package of FIG. 1.

FIG. 4 illustrates a first step of using the blister package of FIG. 1.

FIG. 5 illustrates a second step of using the blister package of FIG. 1.

FIG. 6A is a schematic illustration of another example blister package in an exploded condition.

FIG. 6B illustrates an assembled version of the blister package of FIG. 6A.

### DETAILED DESCRIPTION

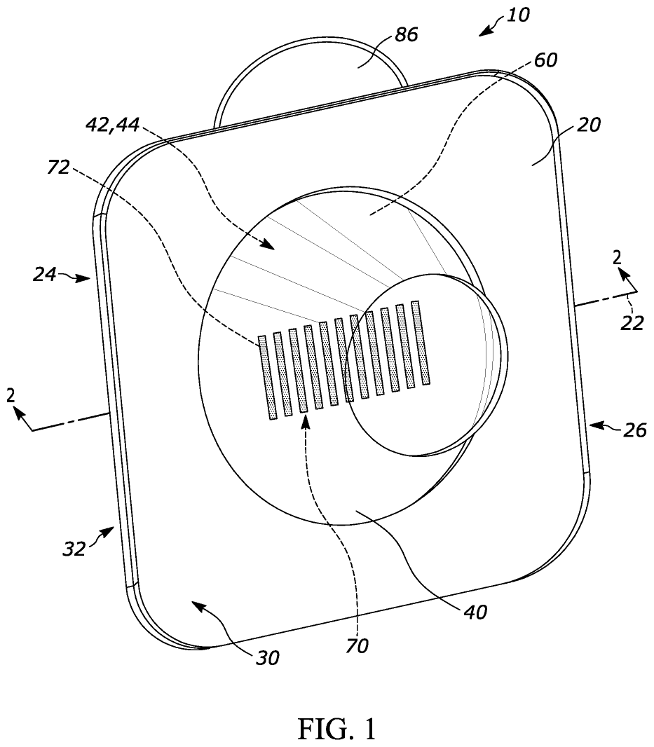

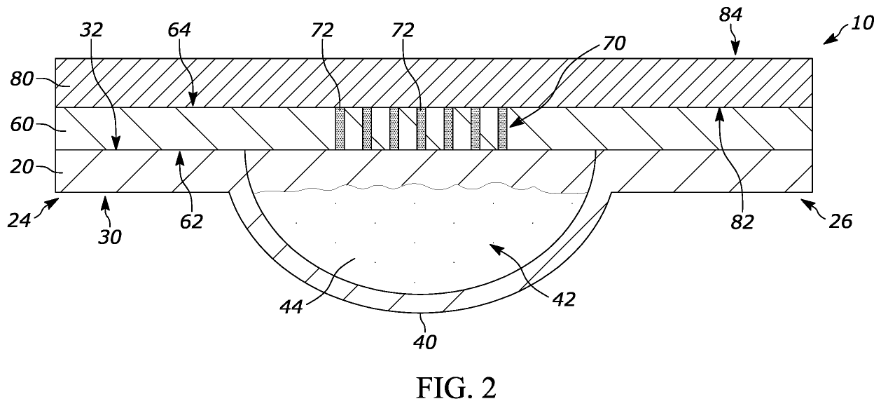

The present invention relates generally to drug packaging and, in particular, relates to unidose ophthalmic blister packs. FIGS. 1-2 illustrate an example blister package 10 in accordance with the present invention. The package 10 includes a base film or layer 20 extending generally along a centerline 22 from a first end 24 to a second end 26. The base layer 20 has a first side 30 and an opposing second side 32. As shown, the base layer 20 is substantially planar, although other non-planar shapes and contours (not shown) are contemplated. The base layer 20 can be formed from a polymer, such as polyvinylchloride (PVC), polyethylene (PE), polypropylene (PP), polyethylene terephthalate (PET) or any other layer material(s) commonly used in pharmaceutical packaging.

At least one dome 40 extends from the first side 30 of the base layer 20. Each dome 40 can be integrally formed with the base layer 20 (as shown) or separately formed and secured thereto (not shown). For example, thermal vacuum forming can be used to pull a portion or portions of a sheet of the base layer 20 into one or more domed shapes such that the domes 40 are integrally formed with the base layer. In multi-dome 40 configurations, the domes can be arranged in a square or rectangular array about the base layer 20.

Each dome 40 has a thickness and configuration that allows it to be elastically deformed relative to the rest of the base layer 20 while remaining connected thereto. Furthermore, each dome 40 has a generally hemispherical shape and defines a cavity 42 for receiving a liquid drug 44. In one example, the cavity 42 can receive a single dose of a liquid, ophthalmic drug 44.

A patterned intermediate film or layer 60 extends over the second side 32 of the base layer 20 and has substantially the same footprint as the base layer. The patterned intermediate layer 60 has a first side 62 abutting the second side 32 of the base layer 20 and a second side 64 facing away from the base layer. That said, the intermediate layer 60 cooperates with each dome 40 to completely enclose the drug 44 within each cavity 42. The intermediate layer 60 can be formed from a polymer, such as PVC, PE, PP, PET or any other layer material(s) commonly used in pharmaceutical packaging. The intermediate layer 60 can be secured to the base layer 20 by, for example, heat seal or an adhesive.

A pattern of nozzle openings 70 is formed in the patterned intermediate layer 60 and arranged in the same pattern in the intermediate layer as the dome(s) 40 are arranged on the base layer 20. That said, each nozzle 70 is aligned with a corresponding dome 40 through the thickness of the base layer 20. The nozzles 70 can be formed as a series of openings or slits 72 arranged in a predetermined pattern, such as parallel to one another as shown. Other patterns, e.g., concentric, aligned or random, and/or shapes, e.g., round openings, are contemplated for the nozzles 70. As will be discussed, the nozzles 70 help to expel the drug 44 within each cavity 42 in a controlled, predetermined manner.

The nozzles 70 can be precisely formed by laser etching the intermediate layer 60 using a laser patterning process on the micro-scale or nano-scale. Lithography can also be used to precisely define the nozzles 70 in the intermediate layer 60. Openings 70 can be in the 10-200 micron range in diameter or cross-sectional width at which point capillary and surface tension forces can dominate the flow of liquid through them. To this end, the nozzle openings 70 can extend from the first side 62 of the intermediate layer 60 to the second side 64 and have a cross-section through the intermediate layer that is very narrow and precisely controlled. Flow through the narrow nozzle openings 70 can be constricted if hydrophobic intermediate layer materials are used.

Typically PP films can be naturally hydrophobic. In this case, hydrophobic surface tension forces do not naturally favor liquid to immediately flow out through the nozzles 70 on its own without applying an added, external force, e.g., squeezing, to the drug 44. In addition, the vacuum back pressure left behind favors retention of the drug. Most drug combinations are aqueous-based and an additional protective layer is therefore desirable to insure water vapor does not escape and cause evaporation of the drug over time. Consequently, a secondary, outer peelable or removable layer 80 is provided over the intermediate layer 60 in a manner that covers all the nozzles 70, thereby helping to prevent evaporation of the drug 44 in the vapor phase.

With this in mind, the removable layer 80 includes a first side 82 abutting the second side 64 of the barrier layer 60 and a second side 84 facing away from the barrier layer. The removable layer 80 has substantially the same footprint as the barrier layer. The removable layer 80 can be secured to the intermediate layer 60 by, for example, heat seal or an adhesive. The removable layer 80 can be a multilayer film formed as a blister foil composite.

More specifically, the removable layer 80 can be formed with aluminum at its core for low water vapor transmission, a thin top layer printable on one side of the core as a protective film (such as PP), and a bottom protective layer (such as PVC) provided on the opposing side of the core. It can also include a release layer such as a PVC or PE film that facilitates peeling of the layer 80 from the intermediate layer 60. A tab or projection 86 is provided on the removable layer 80 and extends laterally beyond the end of the intermediate layer 60.

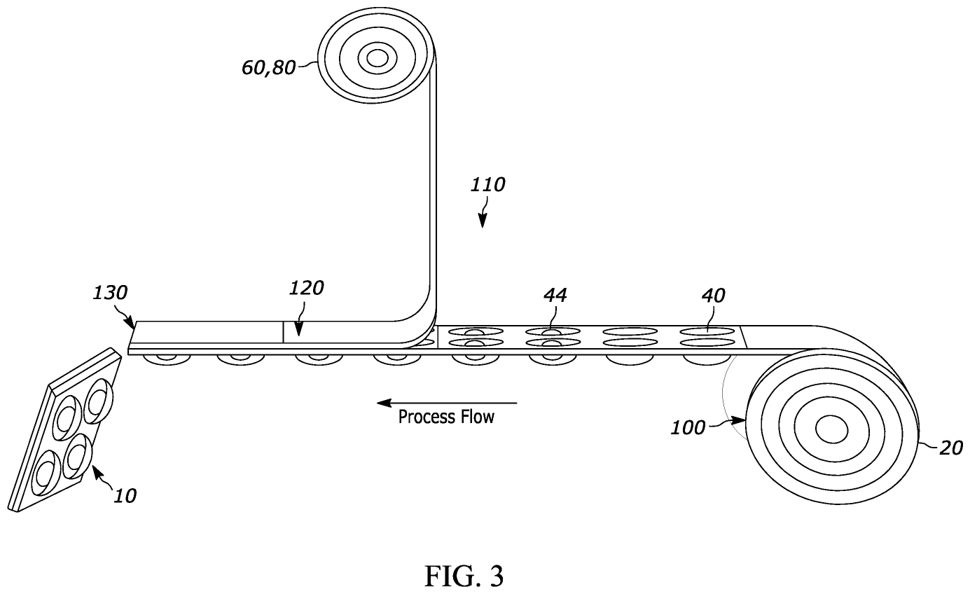

FIG. 3 illustrates an example process for forming the package 10. At step 100, a film is formed by, for example, providing the base layer 20 and separately coextruding the intermediate layer 60 and removable layer 80 together. At step 110, the drug 44 is provided within, e.g., dispensed into, each cavity 42 of the base layer 20. In step 120, the coextruded intermediate layer 60/removable layer 80 is provided over the base layer 20 and secured thereto by, for example, heat sealing. At step 130, individual packages 10 having a predetermined amount of drug 44 dosage are cut from the finished roll. The individual packages 10 can be single dose packages or be configured as a collection of single dose packages arranged in a magazine, e.g., in a strip, ring or the like.

It will be appreciated that the removable layer 80 can already be attached to the intermediate layer 60 and the nozzles 70 formed therein from the bottom side of the two-layer composite in a roll-to-roll process. The two-layer composite can then be cleaned and aseptically treated with ultraviolet light before securing the composite to the base layer 20 to form the whole package 10.

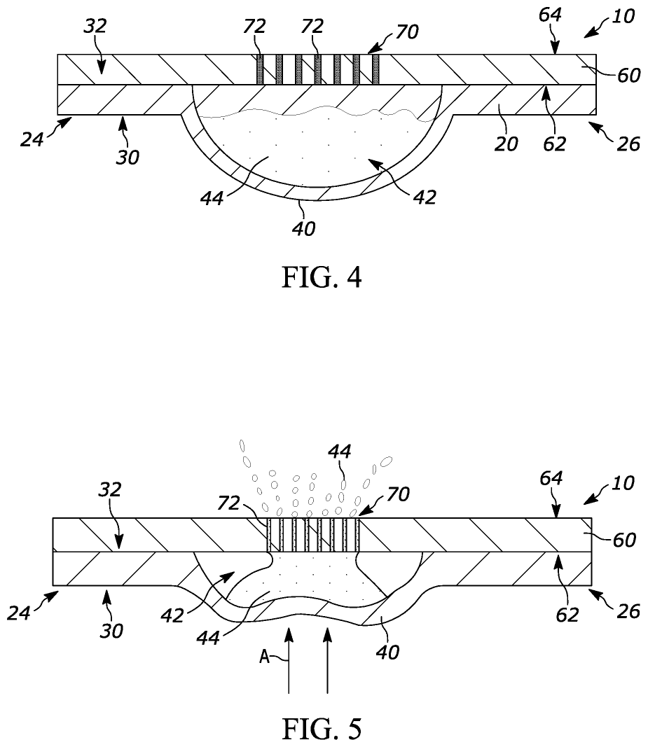

In use, a user grasps the projection 86 and peels the removable layer 80 away from the intermediate layer 60, which exposes the nozzles 70 in the intermediate layer (FIG. 4). Once this occurs, the user can apply force to the dome 40 (indicated at A in FIG. 5) to urge the dome towards and through the intermediate layer 60. This causes pressure within the cavity 42 to increase until the drug 44 is forced through the nozzle 70 and away from the second side 64 of the intermediate layer 60.

More specifically, the pressure increases to a degree sufficient to force the drug 44 through the nozzle 70 to be released as a spray or microstream away from the package 10. It will be appreciated that using many slit openings to form each nozzle 70 allows for a larger dose of more viscous drugs 44 to be easily squeezed out without foregoing surface tension forces that allow for liquid not to spill out immediately if the release layer 80 is removed. In this manner, the slits are hermetically sealed until/unless a force is applied to the dome 40 sufficient to overcome the hermetic seal.

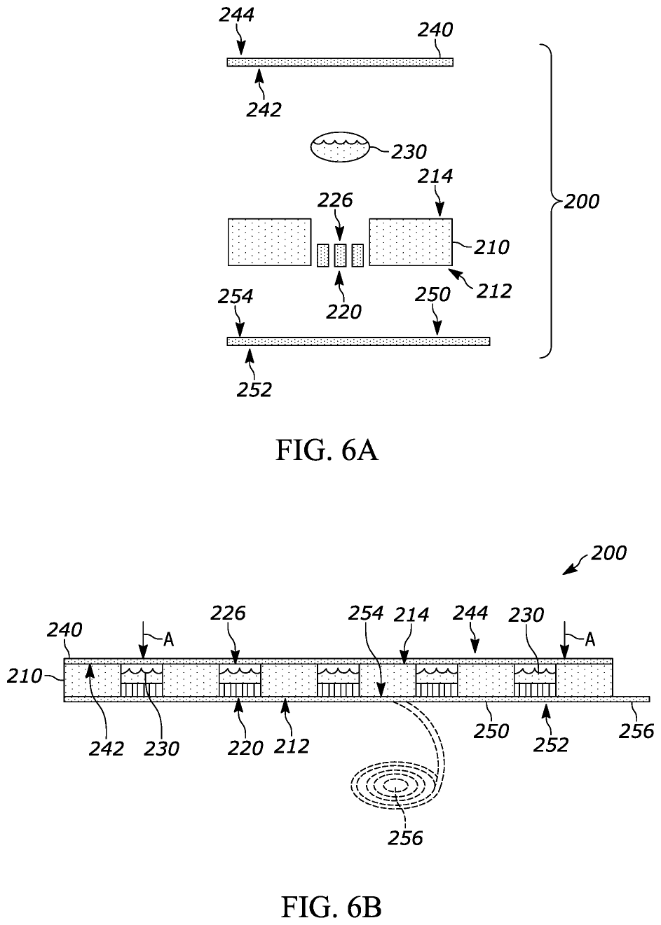

FIGS. 6A-6B illustrates another example blister package 200 in accordance with the present invention. The blister package 200 includes a micro-molded or stamped base or structural layer 210 having a first side 212 and a second side 214. Nozzles 220 are formed in the first side 212 and extend towards the second side 214. The nozzles 220 can be formed as a series of slits or openings.

Cavities 226 are formed in the second side 214 and extend towards the first side 212 into fluid communication with the nozzles. The cavities 226 are aligned with the nozzles 220 through the thickness of the structural layer 210 and there are an equal number of cavities and nozzles. The structural layer 210 can be formed of a polymer and micro-molded to form the nozzles 220 and cavities 226 integrally with layer 210 all in a single step. Each cavity 226 can receive a single dose of a liquid, ophthalmic drug 230.

A first or top seal layer 240 is provided over the structural layer 210 in a manner that covers all the cavities 226. More specifically, the first seal layer 240 includes a first side 242 abutting the second side 214 of the structural layer 210 and a second side 244 facing away from the structural layer. The first seal layer 240 has substantially the same footprint as the structural layer 210. The first seal layer 240 can be secured to the structural layer 210 by, for example, heat seal or an adhesive. Both the structural layer 210 and the first seal layer 240 can be formed from a polymer.

A second or bottom seal layer 250 is provided over the structural layer 210 in a manner that covers all the nozzles 220. More specifically, the second seal layer 250 includes a first side 252 facing away from the structural layer 210 and a second side 254 abutting the first side 212 of the structural layer. The second seal layer 250 has substantially the same footprint as the structural layer 210. The second seal layer 250 can be secured to the structural layer 210 by, for example, an adhesive. The second seal layer 250 includes a tab 256 extending laterally beyond the end of the structural layer 210. The second seal layer 250 can be formed from a peelable, blister foil composite as described above.

In one example, both the first and second seal layers 240, 250 are thermally sealed to the respective sides 212, 214 of the structural layer 210. With this in mind, both sides 212, 214 of the structural layer 210 can be substantially planar to facilitate roll application of the film layers 240, 250 onto opposing sides of the base layer.

Once the package 200 is assembled, the user grasps the tab 256 to peel a section of the second seal layer 250 away from the structural layer 210, which exposes one or more of the nozzles 220 in the structural layer (shown in phantom in FIG. 6B). Once this occurs, the user can apply force as indicated at A to portions of the first seal layer 240 aligned with the cavities 226. In response to the force A, the first seal layer 240 is elastically deformable towards and into engagement with the drug 230.

It will be appreciated that the force A can be applied manually or by using a controlled mechanical strike, such as from a mechanical lever arm or an electromagnetic solenoid strike towards the first seal layer 240. In either case, the force needs to be sufficient enough to urge the drug 230 through the nozzle 220 and away from the side 214 of the structural layer 210 and out past the side 212. To this end, the pressure increases to a degree sufficient to force the drug 230 through the nozzle 220 to be released as a spray or microstream away from the package 200.

The packaging of the present invention advantageously provides a clean, well-defined nozzle for delivering unidose ophthalmic drugs as a spray or microstream in a reliable, consistent manner towards a user's eye. To this end, the removable layer renders the nozzle openings completely unobstructed for drug delivery as compared to existing blister pack constructions that require active puncture of the outer layer, which can result in the broken layer partially obstructing the delivery opening(s). Providing a removable layer is also more repeatable, cheaper to manufacture, and easier to use than the aforementioned puncture-related blister packs.

Furthermore, completely sealed, liquid blister pack cartridges have the potential to be lower cost and less wasteful than blow-fill-seal-fill packaging. Such packages could exist in reel form or as a perforated train that gets dispensed one by one (similar to a Pez dispenser) using an applicator that helps to unpeel the removable layer away from each individual unit. The backside of the blister back can be made of thin polymer that has a bi-stable geometric dome configuration that when struck, for example by a solenoid, will buckle and will project the liquid dose towards an eye by snapping from one state to another to push liquid through nozzles as long as they have a front side supporting plate.

Additionally, providing the removable layer over/covering the nozzles enables the packaging to resist changes in pressure and/or temperature. In other words, the nozzles are sealed to accommodate variations in pressures in airplanes as well as variations in temperatures. The construction also allows the packaging to be preservative-free.

What has been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A blister package for ophthalmic drugs, comprising:
a base layer having a first side and a second side, at least one dome extending from the first side with each dome defining a cavity that receives a liquid drug dose;
an intermediate layer extending over the second side of the base layer and covering each cavity, the barrier layer including an integrally formed nozzle opening aligned with each cavity; and
a, removable layer extending over the intermediate layer and being releasable from the intermediate layer for uncovering each nozzle opening.

2. The package recited in claim 1, wherein the removable layer comprises a blister foil composite including an aluminum core.

3. The package recited in claim 1, wherein the base layer comprises at least one of PE, PVC, PP, and PET.

4. The package recited in claim 1, wherein the removable layer comprises at least one of PE, PVC, PP, and PET.

5. The package recited in claim 1, wherein each nozzle opening is patterned into the intermediate layer.

6. The package recited in claim 1, wherein each nozzle opening is etched into the intermediate layer.

7. The package recited in claim 1, wherein each nozzle opening comprises an array of slits.

8. The package recited in claim 7, wherein the slits extend parallel to one another and can form an extended drop pattern open release.

9. The package recited in claim 7, wherein the slits are between 10-200 μm wide.

10. The package recited in claim 1, wherein the removable layer provides a moisture barrier to the nozzle openings.

11. The package recited in claim 1, wherein the removable layer includes a tab for helping to separate the removable layer from the intermediate layer.

12. The package recited in claim 1, wherein the at least one dome is configured to buckle in response to a force urging the dome towards the intermediate layer to cause the drug to be pushed through the nozzle opening.

13. The package recited in claim 1, wherein each nozzle opening is aligned with the respective cavity through a thickness of the base layer.

14. A blister package for ophthalmic drugs, comprising:
a polymer base layer having a first side and a second side, wherein hemispherical domes extend from the first side with each dome defining a cavity that receives a liquid drug dose;
a polymer intermediate layer extending over the second side of the base layer and covering each cavity, the intermediate layer including an integrally formed nozzle aligned with each cavity and comprising slits extending entirely through the intermediate layer; and
a peelable composite layer extending over the intermediate layer and being releasable from the intermediate layer for uncovering each nozzle.

15. A blister package for ophthalmic drugs, comprising:
a structural layer having a first side and a second side, a nozzle extending from the first side towards the second side and a cavity extending from the second side towards the first side and into fluid communication with the nozzle, wherein the cavity is configured to receive a drug dose;
a first seal layer covering the second side of the structural layer for sealing the drug within the cavity; and
a second seal layer covering the first side of the structural layer and covering the nozzle, wherein the second seal layer is removable from the structural layer to uncover the nozzle, and wherein applying force to the first seal layer towards the nozzle urges the drug through the nozzle and out of the package.

16. The package recited in claim 15, wherein the second seal layer comprises a blister foil composite including an aluminum core.

17. The package recited in claim 15, wherein the nozzle and the cavity are micro-molded with the structural layer.

18. The package recited in claim 15, wherein the nozzle and the cavity are stamped into the structural layer.
